(19) United States
(12) Patent Application Publication
(10) Pub. No.: US 2002/0120297 A1
(43) Pub. Date: Aug. 29, 2002

(54) Vaso-occlusive implants for interventional neuroradiology

(76) Inventor: John H. Shadduck, Tiburon, CA (US)

Correspondence Address: John H. Shadduck, 1490 Vistazo West, Tiburon, CA, 94920, US

(21) Appl. No.: 10/081,383

(22) Filed: Feb. 22, 2002

Related U.S. Application Data
(60) Provisional application No. 60/271,543, filed on Feb. 26, 2001.

(51) Int. Cl. A61N 1/05

(52) U.S. Cl. 607/2

(57) ABSTRACT

A vaso-occlusive implant for use in interventional neuroradiology. The implant body carries a self-contained voltage source comprising nanocrystalline electroactive compositions capable of delivering low levels of electrical energy to body media. The invention provides a system and method for occluding an aneurysmal sac by controlled exposure of a charge over a selected post-implantation interval to activate platelet and other blood compositions thereby resulting in localized thrombogenesis within the vascular malformation.

### CROSS-REFERENCE TO RELATED APPLICATION

This application claims priority from Provisional U.S. Patent Application Ser. No. 60/271,543 filed Feb. 26, 2001 (Docket No. S-ACI-002) having the same title as this disclosure, which is incorporated herein by reference.

### BACKGROUND OF THE INVENTION

1. Field of the Invention

This invention relates to an implant used in interventional neuroradiology, and more particularly to a vaso-occlusive body that carries nanocrystalline electroactive compositions comprising an electrical charge source capable of delivering low levels of electrical energy to body media. The invention provides a system and method for occluding an aneurysmal sac by controlled exposure of a charge over a selected post-implantation interval to activate platelet and other blood compositions thereby resulting in localized thrombogenesis within the vascular malformation.

2. Description of Background Art

A cerebral aneurysm is a common cerebrovascular disorder caused by a weakness in the wall of a cerebral artery or vein. The disorder may result from congenital defects or from preexisting conditions such as hypertensive vascular disease and atherosclerosis, or from head trauma. Approximately 2% to 5% of the U.S. population is believed to harbor an intracranial aneurysm. It is has been reported that there are between 25,000 and 30,000 annual intracranial aneurysm ruptures in North America, with a resultant combined morbidity and mortality rate of about 50%. (See Weir B., Intracranial aneurysms and subarachnoid hemorrhage: an overview, in Wilkins R. H., Ed. Neurosurgry, New York: McGraw-Hill, Vol. 2, pp 1308-1329 (1985)).

Rupture of a cerebral aneurysm is dangerous and typically results in bleeding in the brain or in the area surrounding the brain, leading to an intracranial hematoma. Other conditions following rupture include hydrocephalus (excessive accumulation of cerebrospinal fluid) and vasospasm (spasm of the blood vessels).

One standard form of treating an aneurysm is a microsurgical intervention known as clip ligation of the aneurysm at its base. Long-term studies have established typical morbidity, mortality, and recurrence rates.

The least invasive approach for treating intracranial aneurysms is an endovascular method-which consists of a reconstructive procedure in which the parent vessel is preserved Luessenhop developed the first catheter-based treatment of an intracranial aneurysm (see Luessenhop A. J., Velasquez A. C., Observations on the tolerance of intracranial arteries to catheterization, J. Neurosurg. 21:85-91 (1964)). At that time, technology was not yet developed for successful outcomes. Serbinenko and others deployed latex balloons in intracranial aneurysms (see Serbinenko, F. A., Balloon catheterization and occlusion of major cerebral vessels, J. Neurosurg. 41:125-145 (1974)) with mixed results.

Mullan, et al. reported on the initial series of endosaccular deployment of coils with electrically-induced coagulation and thrombosis (see Mullan S., Raimondi, A. J., Dobben, G., et al., Electrically induced thrombosis in intracranial aneurysms, J. Neurosurg. 22:39-547(1965)). Other approaches also were disclosed, such as deployment of wire elements for direct thrombosis of cerebral aneurysms (see Alksne J. F., et al., Stereotaxic occlusion of 22 consecutive anterior communicating artery aneurysms, J. Neurosurg. 52: 790-793 (1980)). Other investigations involved the use of platinum coils with optional Dacron coverings for treating intracranial aneurysms. However, the morbidity and mortality rates, as well as recanalization and thromboembolic events were still unacceptable at the time of those investigations.

More recently, Guglielmi and colleagues succeeded in developing microcatheter-based systems (GDC or Guglielmi detachable coil systems) that deliver very soft platinum microcoils into an aneurysm to mechanically occlude the aneurysm sac. After the position of the microcoil is believed to be stable within the aneurysm sac, the coil is detached from the guidewire by means of an electrolytic detachment mechanism and permanently deployed in the aneurysm. If coil placement is unstable, the coil can be withdrawn, re-positioned or changed-out to a coil having different dimensions. Several coils are often packed within an aneurysm sac. Various types of such embolic coils are disclosed in the following U.S. Patents by Guglielmi and others: U.S. Pat. Nos. 5,122,136; 5,354,295; 5,843,118; 5,403,194; 5,964,797; 5,935,145; 5,976,162 and 6,001,092

Another distinct manner of treating an aneurysm was disclosed by Gugliemli et al. in U.S. Pat. Nos. 5,122,136 and 5,851,206. In these disclosures, the GDC coil was used to deliver radiofrequency (Rf) to the aneurysm via the guidewire from a remote electrical source. Guglielmi described this particular approach as “electro-thrombosis” in which the conductive guidewire first is used to push the microcoil into the aneurysm, and then used to deliver Rf current to the blood volume in the aneurysm sac to coagulate blood to form an occlusion (see U.S. Pat. No. 5,851,206; Col. 5, line 5). It is believed that such GDC coils were not commercialized due to the risks of creating poorly controlled ohmic blood heating that caused the protein denaturation (or coagulation). For example, such Rf ohmic heating can easily cause a hot spot and rupture the thin aneurysm wall.

Microcatheter technology has developed to permit very precise intravascular navigation, with trackable, flexible, and pushable microcatheters that typically allow safe engagement of the lumen of the aneurysm. However, while the practice of implanting embolic coils has advanced technologically, there still are drawbacks in the use of GDC-type coils. Probably, the principal complications following embolic coil implantation are subsequent recanalization and thromobembolitic events. These conditions are somewhat related, and typically occur when the deployed coil(s) do not sufficiently mechanically occlude the volume of the aneurysm sac to cause complete occlusion. Recanalization, or renewed blood flow through the aneurysm sac, can cause expansion of the sac or migration of emboli from the aneurysm. Recanalization can occur after an implantation of a GDC coil if the COIL does not form a sufficiently complete embolus in the targeted aneurysm. After the initial intervention, the body's response to the foreign material within the vasculature causes platelet activation etc., resulting in occlusive material to build up about the embolic coil. After an extended period of time, the build-up of occlusive material about the foreign body will cease. If spaces between the coils and occlusive material are too large, blood flow can course through these spaces thus recanalizing a portion of the thin wall sac. The blood flow also can carry emboli from the occlusive material downstream resulting in serious complications.

Further, there are some aneurysm types that cannot be treated effectively with an endovascular approach. In such cases, the treatment options then may be limited to direct surgical intervention—which can be highly risky for medically compromised patients, and for patient that have difficult-to-access aneurysms (e.g., defects in the posterior circulation region).

The first type of intracranial aneurysm that cannot be treated effectively via an endovascular approach is a wide-neck aneurysm. In many aneurysms, the shape of the aneurysm sac is shape like a bowler's hat, for example, in which the neck/dome ratio is about 1:1. For the best chance of success in using an embolic coil, an intracranial aneurysm should have a narrow neck that allows the coils to be contained inside the aneurysmal sac. Such containment means that migration of the coil is less likely, and the possibility of thromboembolic events is reduced. To promote coil stability in wide-neck aneurysms, surgeons have attempted to temporarily reduce the size of the aneurysm neck by dilating a non-detachable balloon during coil deployment thereby allowing the coils to engage the walls of the sac while the neck is blocked.

A second type of aneurysm that responds poorly to endosaccular coiling is a giant aneurysm. In these cases, the recanalization rates remain high, the risk for thromboembolic phenomena is high, and the mass effect persists which related to the lack of volume reduction over time.

What is needed, in particular, are vaso-occlusive systems and techniques that reduce the potential for recanalization. Also, systems are needed for endovascular treatment of wide-neck aneurysms and giant aneurysms that can provide acceptable outcomes.

### SUMMARY OF THE INVENTION

The present comprises a vaso-occlusive implant for use in interventional neuroadiology that is adapted to controllably initiate the formation of thrombus in an aneurysm. More in particular, an exemplary implant body of the invention carries thin-layer anode and cathode composition that comprise nanocrystalline electroactive particles coupled to a conductive surface of the implant body. Thus, the implant body the invention provides a self-contained system for providing a positive charge at the surface of a vaso-occlusive implant. The positive charge thus can cause activation of platelets and attraction of negatively charged platelets and other blood compositions to the implant body. A Type “A” system embodiment carries a positive charge source that delivers a particular total electrical discharge over time (rate), which can be continuous or intermittent. This system of the invention is to be contrasted with prior art GDC coils that were disclosed for so-called “electro-thrombosis” in which an in-place catheter carried electrical energy from a remote source to an electrode (GDC coil) within the aneurysm to denature blood proteins thereby causing coagulation.

The invention provides a vaso-occlusive implant body dimensioned for implantation within a vascular malformation such as a cerebral aneurysm that carries a self-contained electrical source.

The invention provides an implant body that carries a volume of nanocrystalline electroactive particles to provide a self-contained voltage source for exposing an electrical charge to body media for therapeutic purposes.

The invention provides a self-contained source for exposing a positive electrical charge to platelets within blood flow within an aneurysm to activate the platelets—thereby initiating thrombogenesis.

The invention provides a self-contained source for exposing a positive electrical charge at an exterior of the device to attract negatively charged blood compositions, in particular platelets and fibrinogen, to rapidly cause thrombus formation in an aneurysm.

The invention advantageously provides a method for charge-induced occlusion of a vascular malformation that does not rely on packing the malformation with embolic materials.

The invention provides a system and method that causes rapid formation of thrombus substantially without risk of perforating the wall of the aneurysm.

The invention provides a method for occluding a vascular malformation that is a non-thermal process relying on charge-induced or charge-enhanced thrombogenesis formation that utilizes very low levels of electrical activity in body media.

The invention provides a system and method that allows instantaneous detachment of a vaso-occlusive implant by using photoabsorption to create a spall plane across a bond between the implant body and a guiding member.

The invention provides a system and method that allows for non-thermal de-coupling of an implant body from a guidewire member.

The invention advantageously provides a system and method that causes rapid formation of thrombus without denaturing proteins in blood by ohmic heating of blood in an aneurysm.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout this disclosure.

FIGS. 1A-1B are images of platelets in a resting state and in an activated state.

FIG. 2 is a graphic illustration of a platelet showing receptors and other aspects of its cytoplasm.

FIG. 3 is an illustration of an endovascularly introduced microcatheter that carries an exemplary Type “A” vaso-occlusive implant body toward a typical narrow-neck aneurysm suitable for treatment with the implant.

FIG. 4 is a graphic illustration of an exemplary Type “AA” vaso-occlusive implant body deployed in the narrow-neck aneurysm of FIG. 3.

FIG. 5 is an enlarged cross-sectional view of an element of the implant body of FIG. 4 taken along line 5-5 of FIG. 4. showing an exemplary arrangement of anode and cathode layers comprising components of the charge source in accordance with the principles of the invention

FIG. 6 is a schematic longitudinal sectional view of the implant body of FIG. 4 showing an exemplary arrangement of conductor elements coupled to the interior anode and cathode compositions.

FIG. 7A depicts the interior shape of a typical “bowler hat” or wide-neck aneurysm that is treatable with a Type “B” embodiment of the invention.

FIG. 7B depicts the interior shape of the wide-neck aneurysm of FIG. 7A that indicated the objective of the Type “B” embodiment in providing an implant that engages and slightly bulges the aneurysm wall above the neck.

FIGS. 8A-8D depicts the deployment of a Type “B” shape-memory vaso-occlusive implant body; FIG. 8A being an initial step in deployment of the implant body; FIG. 8B being the implant body further deployed wherein release of tension cause lateral projection and un-twisting of the implant body; FIG. 8C being deployment of the implant body in the aneurysm sac prior to de-coupling from paired guide members; and FIG. 8D showing the final deployed implant body in the aneurysm sac after de-coupling when the positive charge source induced thrombogenesis.

FIG. 9 shows the “spall plane” detachable coupling system of the invention.

FIGS. 10A-10C show the wide-neck aneurysm of FIGS. 8A-8B from its exterior and optional manners of deploying one or more Type “B” vaso-occlusive implants; FIG. 10A being the aneurysm prior to deployment of an implant; FIG. 10B being the aneurysm after deployment of a single implant body oriented in alignment with the axis of the parent vessel; and FIG. 10C being the aneurysm after deployment of a two implants—one oriented in alignment with the vessel axis and one generally transverse to of vessel axis.

FIG. 11 is view of an alternative Type “B” implant body of shape-memory material is a final deployed condition wherein a grid of shape memory elements extend across the neck of the aneurysm.

FIGS. 12A-12B are plan and sectional views of an alternative implant body having a flexible outer sleeve and carrying flexible layers of electroactive compositions.

### DESCRIPTION OF THE PREFERRED EMBODIMENTS

I. Principles of the Invention Relating to Charge-Induced Thrombogenesis

The objective of the present invention is to controllably initiate thrombogenesis within in a targeted aneurysm sac or other vascular malformation. The term thrombosis and thrombogenesis, as used herein, relate to the activation and aggregation of blood factors—the initial cascade of events relating to platelet and fibrin actions that later lead to an entrapment of cellular elements and ultimately to an occlusion of the vasculature. Controlled thrombosis is used by the method of the invention to create an obstruction to blood flow with a portion of a patient's vasculature at the point of thrombus formation.

To understand the mechanisms of action underlying the method of the invention, it is necessary to describe the role of platelets in thrombosis—and the manner in which the devices and techniques of the invention target platelet activation. The principal platelet functions relate to activation, adhesion, recruitment and aggregation, all of which are controlled by the activity of platelet membrane receptors, which biochemically are glycoproteins (GP). For the purposes of the present invention, the more specific objective is temporal control of platelet activation over a selected time interval that extends well beyond the time of the endovascular intervention and device implantation.

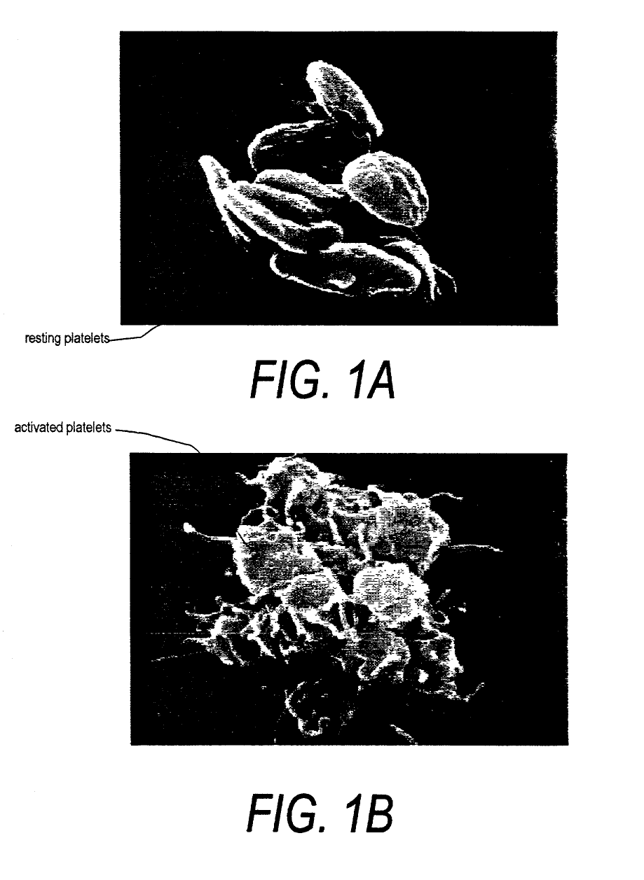

As shown in FIG. 1A, platelets or blood thrombocytes are the smallest corpuscular components of human blood and have a diameter of about 2-4 μm, with numbers varying from 150,000 to 300,0001 mm³ of blood. Platelets are not cells since they have no nucleus, but rather are cytoplasmic fragments of megakaryocytes. The origin of platelets is the bone marrow, where megakaryocytes—the result of mitotic proliferation of a committed progenitor cell—liberate platelets as the end product of protrusions of their membrane and cytoplasm. The typical shape of resting platelets is discoid (FIG. 1A) and upon activation they undergo a shape change to a globular form with pseudopodia up to about 5 μm in length (FIG. 1B). Platelet activation can occur when injury to a vessel wall exposes sub-endothelial components, especially collagen, to the platelet receptors. After platelets are activated, they adhere to the damaged area and become cohesive to other platelets. This platelet aggregation leads to the formation of a platelet plug, which can prevent blood loss through a vessel rupture and allows the vascular reparative process to begin.

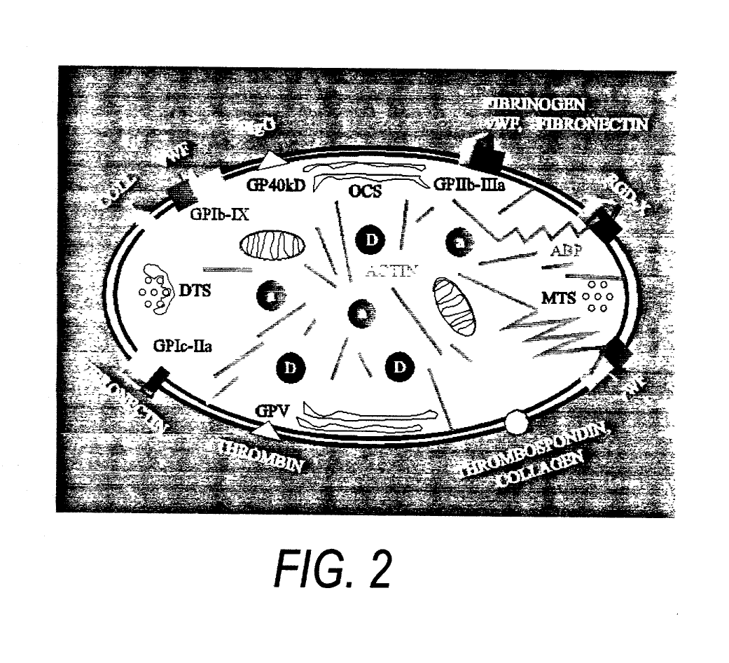

Platelet exterior membranes consist of a typical phospholipid bilayer of membrane. Embedded in this structure are different kinds of glycoproteins (GP) that serve as receptors for activation and interaction with other cells. FIG. 2 is a graphical illustration of a platelet, identifying several receptors, cytoskeleton proteins, microtubular system (MTS), etc. Most important for the purposes of this disclosure (as will be described below) is the glycoprotein receptor GPIb/IX that mediates platelet adhesion to subendothelial collagen via von Willebrand factor (vWf)—as well as being responsible for a negative charge at the platelet surfaces. Another glycoprotein, GPIIb/IIIa, serves as the binding site for adhesive molecules, for example, fibrinogen, vWf, and fibronectin. GPIIb/IIIa therefore permits the intercellular interaction between platelets or between platelets and other cells. For the majority of GP-complexes, the connections to the cytoskeleton have been identified—comprising actin (10-20%) and myosin (15-20%) proteins that form a three-dimensional network through the platelet cytoplasm. Another two-dimensional network of shorter actin fibers serve as a membrane skeleton, responsible for the discoid shape of the resting platelet. Another bundle of microtubules (MTS) supports the actin membrane skeleton to maintain the discoid shape. In the periphery of the cytoplasm, another membrane system is called a dense tubular system (DTS), named according to the inherent electron opacity. Surrounding the organelle zone is a membrane system with invaginations of the platelet's plasma membrane (OCS) and offers additional membrane capacity during activation, when the surface-to-volume ratio increases through the membrane's extroversion into pseudopodia.

Organelles are somewhat evenly distributed in the cytoplasma of resting platelets. Mitochondria serve as the energy source, since resting platelets cover their energy expenditure by oxidative phosphorylation, similar to other cells. The largest number of organelles are storage granules (˜40/platelet) containing fibrinogen, thrombospondin, FV, von Willebrand factor, beta-thromboglobuline (β-TG), platelet factor 4 (PF4), etc.

Upon activation, platelets release their granula contents, contributing to diverse interactions with other platelets and other cells, which initiates thrombogenesis—including platelet adhesion, recruitment and aggregation. Platelet adhesion to subendothelial collagen is mediated via vWF by the trans-membrane complex of GPIb-IX. This glycoprotein also is responsible for the negative charge of platelet surfaces—and serves as a specific target of the method of the invention.

Of particular interest, the devices and techniques of the invention relate to systems for providing a positive charge at the surface of a vaso-occlusive implant, in several related embodiments. The positive charge source thus can cause activation of platelets to by attracting the platelet toward the implant body and thereafter, it is believed, interacting with GPIb-IX.

In a first form of Type “A” device, the vaso-occlusive implant carries a self-contained charge source that can expose a charge from a positive polarity surface conductor at the exterior of the implant body for an extended time interval following implantation of the device (and removal of the microcatheter system). The positive charge about the surface of the implant body can attract and activate platelets to thereby initiate thrombogenesis. In another form of Type “A” device, the implant body itself carries both positive and negative polarity surface portions, which can provide very low level current flow within the aneurysm sac—which again will activate platelets and, it is believed, will enhance thrombogenesis. These Type “A” systems are to be contrasted with prior art devices that were disclosed Guglielmi and others for so-called “electro-thrombosis” in which ohmic heating of blood was caused by high Rf current densities in order to denature proteins in blood—thereby causing true coagulation (i.e., protein denaturation). The Type “A” systems disclosed herein provide levels of electrical energy that are well below the levels that cause ohmic heating of blood.

II. Construction and Method of Use of Exemplary Type “A” Embodiment

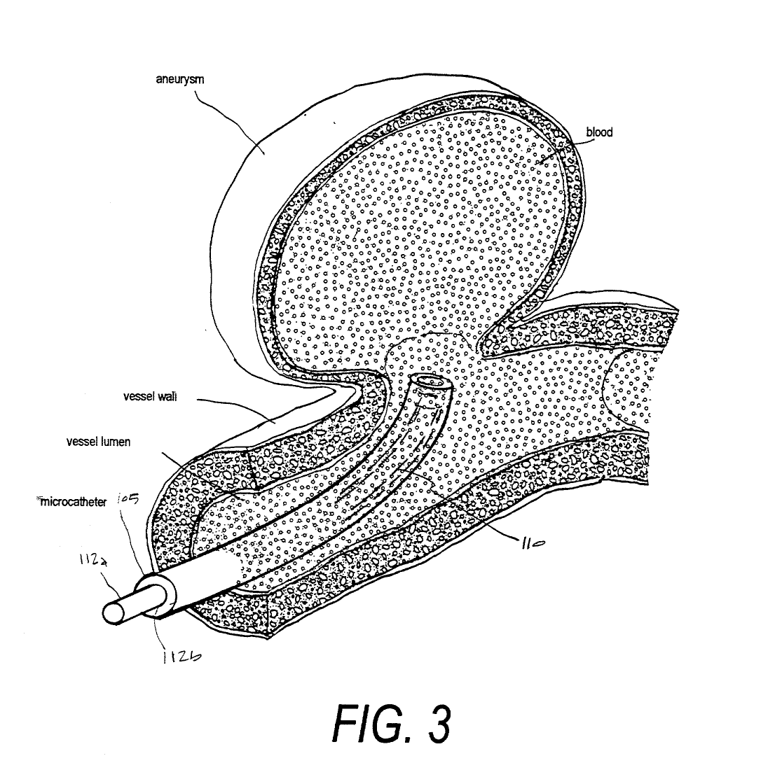

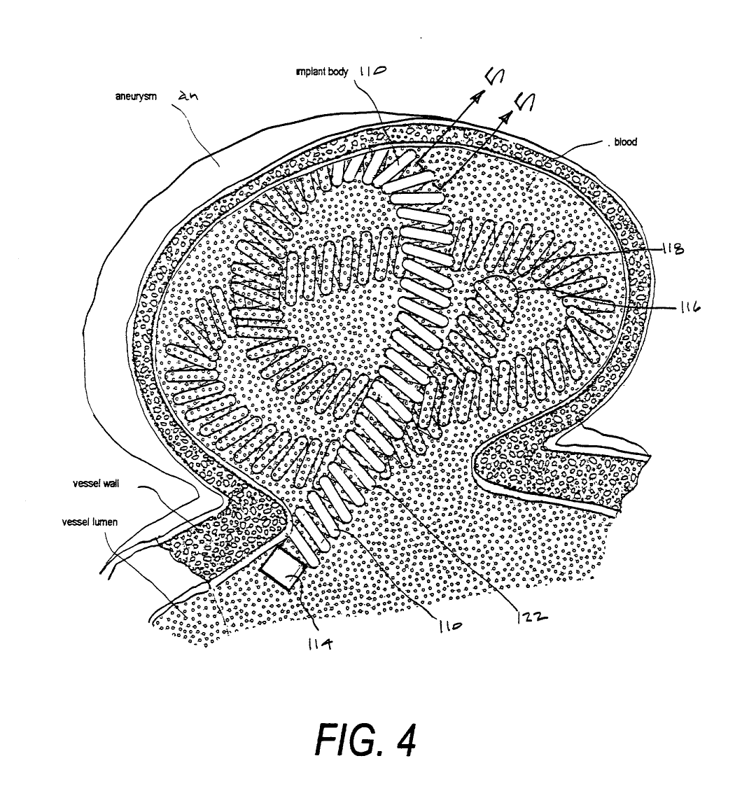

FIGS. 3 & 4 show elevational views of the distal end of a microcatheter 105 that is adapted to carry a Type “A” vaso-occlusive implant body 110 into an intracranial aneurysm an. In FIG. 3, the elongate implant coil or body 110 is detachably coupled to guidewire 112a and carried in catheter lumen 112b. The detachable coupling between the implant body and guidewire may be any type known in the art, such as an electrolytic coupling, a mechanical release coupling, etc., which coupling is indicated generally at 114. Later, in this disclosure, a novel “spall plane” de-coupling system will be described, but such a de-coupling system is not a necessary component of this Type “A” embodiment. FIG. 4 shows an exemplary embodiment of implant body 110 that comprises an elongate wire-type member having a core structural element 115 wound in a coil that can be pushed into the aneurysm sac, similar in form to coils known in the art. The implant body 110 has a length, coil cross-section and wire diameter suited for microcatheter delivery and endosaccular implantation, for example with lengths ranging from about 2 cm. to 50 cm., coil diameters ranging from 0.01″ to 0.10″, and wire diameters from about 0.001″ to 0.01″. The distal end 116 of implant body 110 has tip structure 118 that is dull, soft, or rounded so as to prevent the distal end from rupturing a vessel wall as it is pushed into the aneurysm sac.

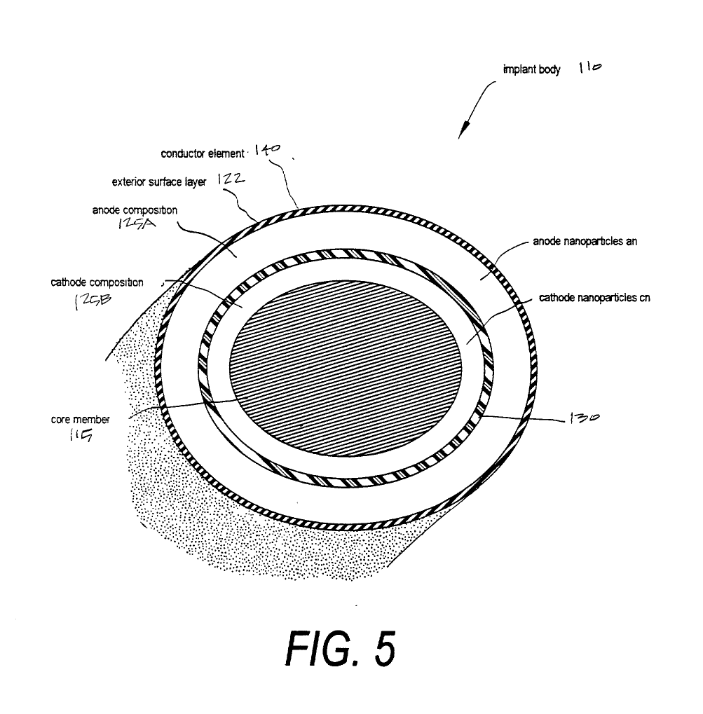

The implant body 110 corresponding to the invention carries a self-contained electrical potential source 120 (or voltage source) that is utilized to provide a low level positive electrical charge about an exterior 122 of the implant body 110 to cause rapid platelet attraction and activation, as well as the attraction and aggregation of other blood compositions that are believed to carry at least a transient negative charge (e.g., red blood cells, fibrinogen). The electrical source 120 of the invention is more particularly shown in FIG. 5, wherein the greatly enlarged sectional view of wire-type member of the body 110 shows a number of layers about the structural core wire 115. In one embodiment, the wire core 115 has round cross-section but may also have any oval, flattened, rectangular or polygonal cross-section.

In general, a Type “A” implant body 110 defines interior portions that carry thin layers of an anode composition (indicated at 125A) and a cathode composition (indicated at 125B) separated by a separator layer 130. The exterior of implant body 110 comprises other thin layer depositions, with a metallic conductive surface layer indicated at 140 and a non-conductive layer indicated at 142. Of particular interest, the anode and cathode composition layers 125A and 125B comprise volumes of electroactive particles, hereafter more particularly identified as anode nanoparticles anp and cathode nanoparticles cnp, respectively (see FIG. 5). In one aspect of the invention, the cathode composition comprises cathode nanoparticles cnp having an average diameter less than about 2000 nm. Preferably, the cathode nanoparticles cnp have an average cross-section or diameter ranging from about 1.0 nm to 500 n. More preferably, the cathode nanoparticles cnp have an average cross-section ranging from about 1.0 nm to 250 nm. As will be described below, the optimal electrical charge range (or voltage range) required for the method of the invention provided by source 120—of which the cathode composition comprises a first component—can be deposited together with a binder material 144 in a thin layer about the surface of the underlying structural wire 115 of the implant body. The thin layers or coatings preferably have a thickness ranging from about 0.25 μm to 50 μm. More preferably, the layers are from about 0.5 μm to 10 μm. The separator element 130 in this embodiment is a thin deposition of any polymer electrolyte or porous polymeric material known in the art of voltage source design and has a thickness similar to that of the electroactive layers or coatings.

The thin depositions or coatings that comprise the electroactive layers have meaningful electrical discharge capabilities since the electroactive anode and cathode compositions comprise nanoparticles that provide correspondingly increased particle surface areas. One preferred process for manufacturing electroactive particles is a laser pyrolysis method developed by NanoGram Corporation, 46774 Lakeview Blvd., Fremont, Calif. 94538. NanoGram describes its laser pyrolysis process method as a “Nano-Particle Manufacturing” (NPM™) system. The process uses a laser-driven non-equilibrium chemical reaction process in which gases are combined to form simple or complex nanoscale compounds. Aspects of this process are disclosed in U.S. Pat. No. 5,958,348 assigned to NanoGram Corp., which patent is incorporated herein by this reference. NanoGram Corporation's processes are capable of building nanoscale particles from the atomic level to allow for precision particle sizes and particle purity. Particles suitable for electroactive anode and cathode layers also can be made by other manufacturing processes, such as by a controlled reaction vessel or by a machine grinding process. One of the potential uses for nanoscale particles identified by NanoGram relates to use in voltage sources.

The description above characterizes the dimensions, uniformity and purity of nanocrystalline electroactive materials that enable the invention. The scope of the invention includes the use of any materials known in the art of voltage sources for fabrication of the anode and cathode nanoparticles anp and cup, as well as the separator element 130 to provide a self-contained voltage source 120 and in one embodiment can be vanadium oxide particles. Such vanadium oxide particles are known in the art and can be provided in the nanometric scales referred to above. Nanoscale vanadium oxide particles can be produced in varied oxidation states and crystalline structures. U.S. Pat. No. 6,106,798 assigned to NanoGram Corp., disclosed methods of producing such nanometric dimensioned vanadium oxide particles. The use of such particles is best suited for implants that have a substantially thick permanent non-corrosive exterior layer, such as titanium or titanium alloy, that can be exposed to contact with body media indefinitely. Other suitable electroactive nanoscale particles that are non-reactive to the implant environment—and thus biocompatible in the event of corrosion of exterior layers of the implant—can be characterized and fabricated by NanoGram Corp., and may include carbon and zinc compositions together with an acidic electrolyte separator element; silver-zinc electroactive compositions; manganese oxide and zinc compositions with an alkaline separator element; as well as any other similar biocompatible compositions known in the art of electroactive materials and electrical charge storage. In implant bodies that have exterior coating that are proven to be non-corrodible, the anode and cathode nanoparticles can be any lithium transition-metal oxide. For example, lithium oxide may be suited for use as the anode electroactive material when carried at an interior coating of implant body 110. The binder, for example, can be polyethylene, polypropylene, polytetra-fluoroethylene or mixtures or co-polymers thereof. For example, the use of these electroactive materials are known in the art and described in U.S. Pat. No. 5,958,348 assigned to NanoGram Corp.

The separator element 130 is substantially electrically insulative and provides for passage of at least some types of ions therethrough. Such ionic transmission through separator element 130 can provide for electrical neutrality in the varied sections of the voltage source 120. The separator element 130 prevents the electroactive particles of anode composition anp from contacting electroactive particles of the cathode composition cnp. A preferred material for separator 130 can be any of the polymers described above that are suitable for use as a binder 144 in the anode and cathode layers. Such polymer separator element can be porous to provide for ionic conduction. As an alternative, such polymer separators can comprise a solid electrolyte formed from a polymer such as polyethylene oxide. In this case, such a solid electrolyte separator element 130 incorporates an electrolyte into the polymer matrix to provide for ionic conduction without the need for fluid-type transmissions.

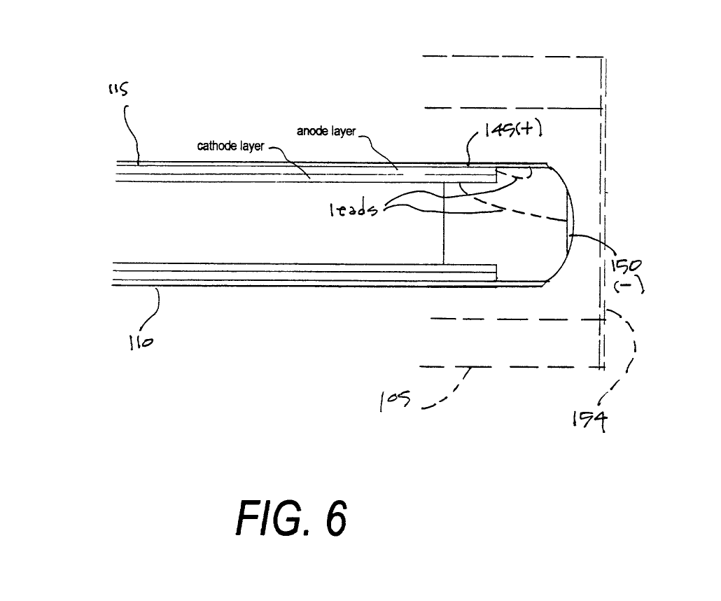

To expose an electrical charge to blood flow within an aneurysm, the body 110 carries at least one exposed conductor at the exterior of the implant body, each such conductor coupled to respective anode and cathode layers, as shown schematically in FIG. 6. Since a principal proposed mechanism of action is to activate platelets by exposure to a positive charge over a selected post-implantation time interval, the exterior non-corrosive layer also comprises an exposed positive (+) conductor 145 (or electrode) to provide the maximum exposure to body media. While this embodiment provides positive (+) conductor 145 that covers substantially all of the exterior surface of the implant body 110, this exposed conductor can also be provided only along or about selected portions of the implant body, as will be described further below. The implant body 110 optionally carries an exposed negative (−) conductor 150 that in this embodiment is at an end portion of member 115 as shown in FIG. 6. The conductor 150, if exposed, is used in a method of the invention described further below to cause a very low current density in the aneurysm to further activate the body's wound healing response within the aneurysm sac. FIG. 6 also shows that the distal termination of the catheter 105 carries a removable, or perforatable, insulative cover 154 to prevent contact of implant body 110 with the environment prior to use which could discharge the electrical energy carried by the implant. This cover 154 is removed just before use, or a very thin cover may be provided that can be pushed through after endovascular deployment of the working end.

By altering the average thickness or cross-sectional dimensions of the anode and cathode layers, as well as the conductive characteristics and dimensions of anode and/or cathode conductor portions 145 and 150 (see FIGS. 5 & 6) exposed at an exterior of implant body 110, an electrical discharge profile and capacity provided by the charge (voltage) source 120 can be modeled. The most useful manner of identifying the electrical charge delivery capability of the implant body 110 is to assume that exposed conductor portions 145 and 150 are provided at an exterior of the device and measuring actual voltage. In this case, the anode and cathode layers of implant body 110 preferably would provide a voltage ranging between about 0.01 volts and 5 volts. More preferably, the voltage would range between 0.25 volts and 3 volts.

To provide the exterior conductive coating 140 on implant body 110 as shown in FIG. 5, an electroless plating process known in the art can be used to deposit the conductor layer on the underlying structural element 115 and the electroactive layers. The thickness of any conductive coating 140 can range from about 0.001″ to 0.01″ and consist of titanium, or any other biocompatible material (e.g., gold, platinum, silver, palladium, tantalum, tin or combinations or alloys thereof). Other potential manners of depositing a thin conductive layer on body 110 are laser reactive deposition processes, plasma enhanced chemical vapor deposition (PECVD) processes and electron beam deposition processes as are known in the art. The polymer insulative layer 142 can be deposited on implant body by any deposition or coating process known in the art, for example by any medical device coating manufacturer, such as SurModics, Inc., 9924 W. 74^{th} Street, Eden Prairie, Minn. 55344.

The implant body 110 further carries radio-opaque marker portions as are known in the art to allow imaging of the implant 110 as it is fed into the targeted anerysmal sac. The radio-opaque marker portions typically would comprises a marker 160a and 160b around each end of body 110 (see FIG. 4), a marker at the distal end of the microcatheter, and/or or an elongate marking along the length of the implant body.

Now turning to FIGS. 3, 4 & 8A-8D, the methods of the invention (or charge-induced platelet activation methods) are graphically depicted in the occlusion of an intracranial aneurysm an. The physician removes the insulator cover 152 at the distal end of the catheter (see FIG. 6). FIGS. 34 depict the physician introducing the distal working end microcatheter 105 through lumen 178 of vessel 180 to the targeted site, for example in a transfemoral approach as is known in the art. In this case, blood is indicated at 185 and comprises the body media that occupies the aneurysm an. After the working end of the catheter is positioned adjacent to, or partially within, the aneurysm an, the physician pushes a guidewire 112b that is coupled to implant body 110 to feed the implant into the aneurysm sac. Any length implant body can be selected depending on the estimated volume of the aneurysm, all under flouroscopic viewing. After the implant body 110 is stabilized, the implant is decoupled from guidewire 112b by electrolysis of the sacrificial coupling 114 or by other means known in the art. Next, the physician removes the microcatheter and closes the endovascular access site.

As represented generally in FIG. 3, the volume of aneurysm an need not be completely packed with the implant body 110 corresponding to the invention. This is to be contrasted with the use of GDC embolic coils as described in the Section above titled Description of the Background Art. In a typical case utilizing such embolic coils, the physician feeds multiple coils into the aneurysm since the objective is attain substantially complete mechanical occlusion of the aneurysm volume with a foreign material. In contrast, the implant 110 of the invention utilizes its self-contained charge source 120 to expose a positive charge about the surface of conductor 145 which thereby activates and attracts negatively-charged platelets toward the implant, as well as negatively charged red blood cells, white blood cells, etc. The electrical charge capacity of source 120 is designed to provide the amount of energy required to accomplish the method of the invention, which can be a time interval ranging from about 1 minute to 120 minutes. As described previously, the implant body 110 can expose a positive charge conductor 145 to body media within the aneurysm, or the implant body 110 can expose a both positive and negative polarity conductors 145 and 150 to blood to cause low level current flow between the spaced apart conductors to perform the method of the invention.

III. Construction and Method of Use of Exemplary Type “B” Embodiment

The Type “A” embodiment disclosed a “basic” preferred embodiment of the invention that is adapted for treatment of a typical narrow neck aneurysm. Further, the Type “A” implant body is deployed in substantially a two dimensional state which can conform to the aneurysm sac into three dimensions by twisting as it is pushed inwardly. The Type “B” embodiment can be used in any type of aneurysm, but has features that make it suited for endovascular treatment of wide-neck aneurysm and giant aneurysms. Also, the Type “B” embodiment is of shape memory material that provides a three dimensional shape to the implant body when deployed. The Type “B” implant body combines several independent and distinct features, any one of which can also be combined with the electroactive aspects of the Type “A” embodiment.

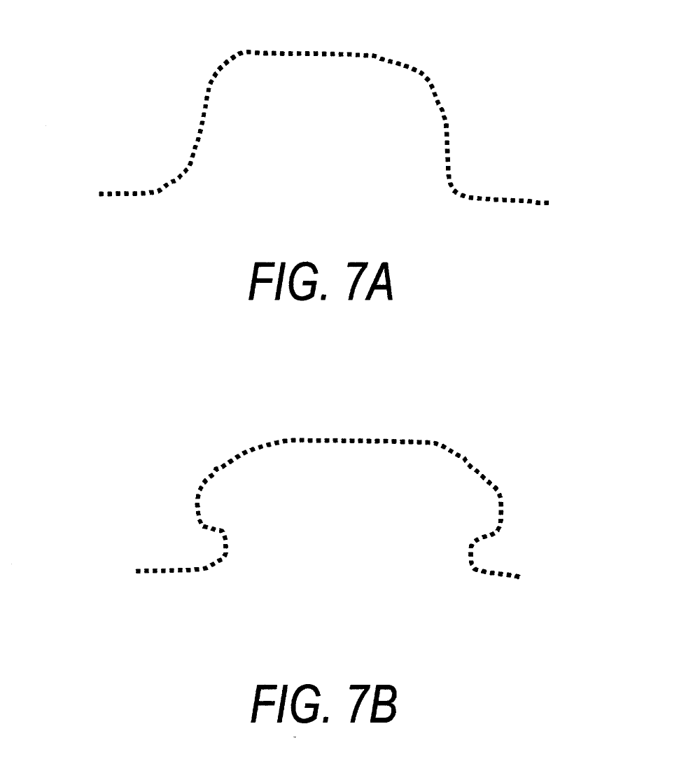

The principal underlying the Type “B” implant body for treating a wide neck-aneurysm is illustrated in FIGS. 7A-7B. In FIG. 7A, a typical “bowler hat” or wide-neck aneurysm is shown which defines a neck dimension nd and a dome diameter dimension dd that have a ratio of about 1:1. The height dimension hd of the aneurysm typically is also similar to the dome dimension dd. As shown in FIG. 7A, the aneurysm sac has a neck n, a dome d (or fundus) and a rim wall portion rw. The objective of the invention is to provide an implant body 210 of a shape memory material, preferably having three dimensions in a deployed state, that can deform the aneurysm sac as shown in FIG. 7B by pushing outwardly portions of the rim wall rw while at the same time slightly collapsing the dome d downwardly (see arrows in FIG. 7B). Stated another way, the invention is adapted to deform the “bowler hat” aneurysm into a cross-sectional shape more like a typical narrow-neck aneurysm that will contain an implant body.

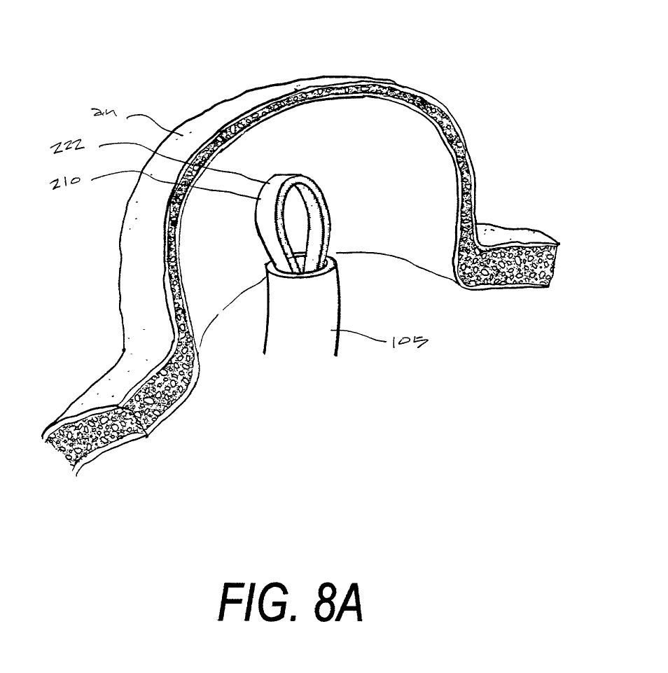

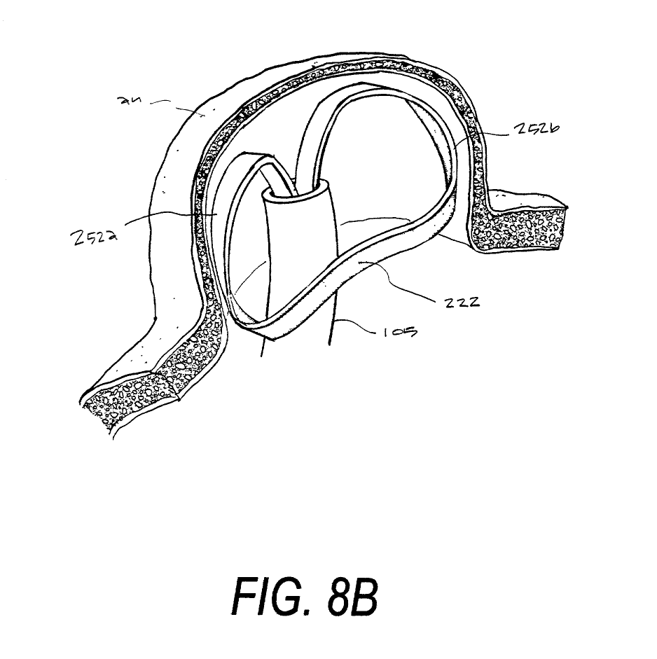

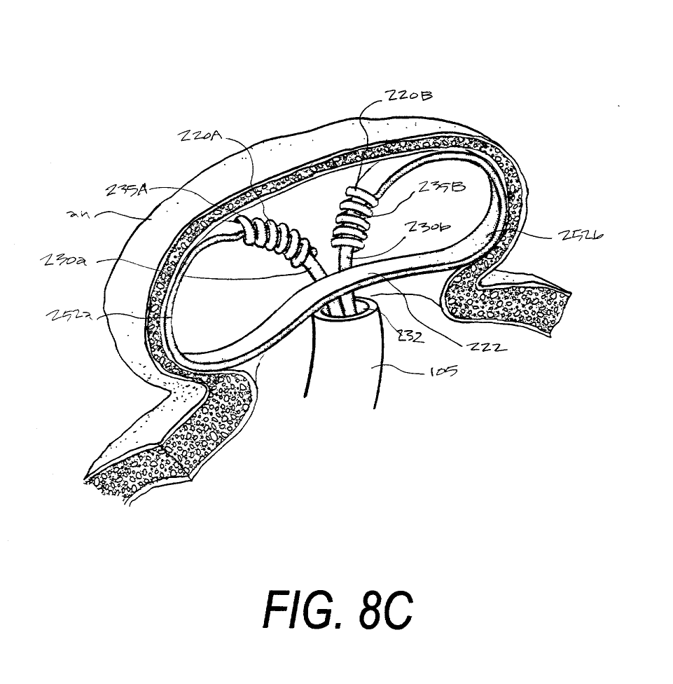

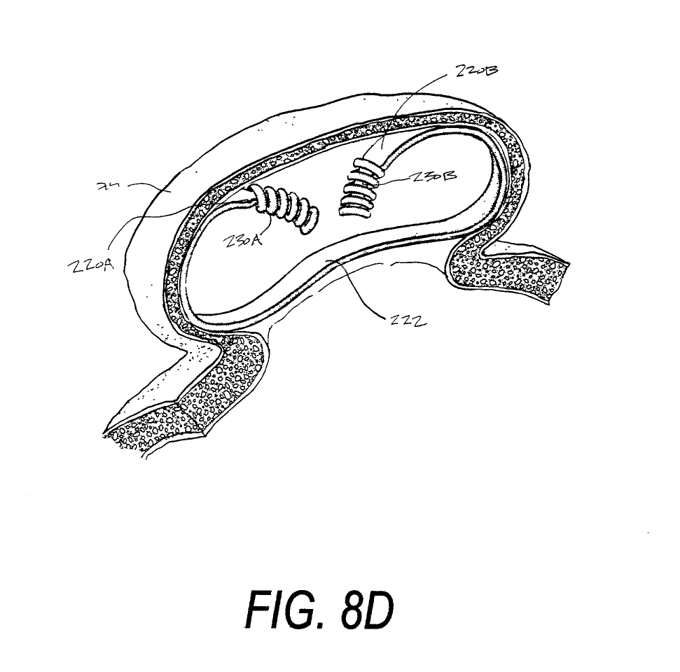

FIGS. 8A-8D show an exemplary Type “B” implant body in its deployment from a first collapsed or linear shape (as when carried by catheter) to and second expanded shape after being deployed from catheter 205. The dashed line in FIGS. 8A-8D indicate the interior wall of the aneurysm an as the implant body is deployed.

As can be best seen in FIGS. 8C and 8D, the implant body 210 comprises an extending member 214 that extends between first and second end portions 220A and 220B. The implant member defines a medial portion indicated at 222 that extends between the first and second end portions 220A and 220B. In this exemplary embodiment, the medial portion 222 is a flattened ribbon-type member of a shape memory material that forms itself into a pre-determined shape, such as a nickel titanium alloy described in U.S. Pat. No. 5,645,558, which is specifically incorporated herein by reference. In FIGS. 8A-8D, the medial portion 222 of the implant body 210 is shown as a flat rectangular member to better illustrate the rotational twisting of the implant body as it moved to the (repose) second expanded shape from the (tensioned) first collapsed linear shape. It should be appreciated that the medial portion 222 of implant body 210 also can be round, oval or any other cross-section.

FIG. 8D shows that the first and second end portions 220A and 220B of implant body 210 are detachably coupled to the distal ends of first and second slidable guide members 230a and 320b that are carried in lumen 232 of catheter 205. Each of the first and second end portions 220A and 220B terminate in detachable coupling portion that couples the guide members 230a and 320b to the implant body. These coupling portions can comprise an electrolytic sacrificial coupling, a mechanical coupling or any other coupling known in the art of GDC coils. Of particular interest, however, the present invention provides a detachable coupling that comprises a stress confinement (or spall plane) detachment system that utilizes photonic energy for instantaneous detachment of the implant body 210 from the guide members 230a and 230b.

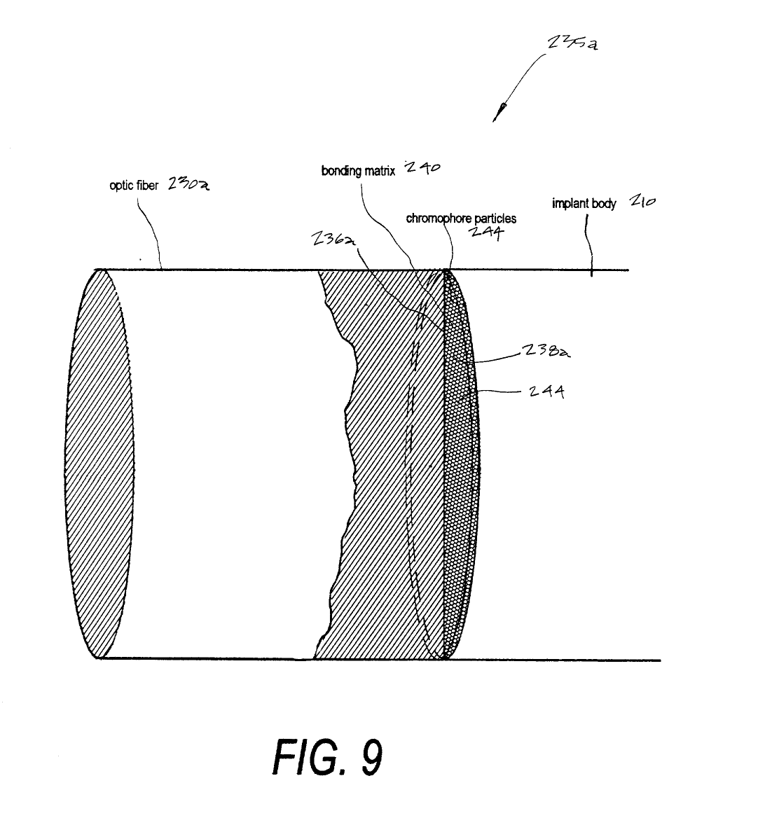

One such stress confinement coupling portion 235a is shown in more detail in FIG. 9. FIG. 9 shows that that guide members 230a is a flexible optic fiber having a diameter ranging from about 100 μm to 1 mm. that has terminal surface 236a. The coupling portion 235a has a terminal surface indicated at 238a that interfaces with the terminal surface 236a of the optic fiber. These two terminal surfaces 236a and 238a are connected by bond matrix 240 that comprises (i) a volume of nanocrystalline particles 244 that have a selected absorption coefficient μₐ (cm⁻¹) to cooperate with a selected wavelength (λ) that can be delivered through the optic fiber guide member, and (ii) a binder composition 246 that is suited for bonding the nanocrystalline particles 244 and terminal surfaces 236a and 238a together in a thin layer connection. The binder composition, for example can be any biocompatible cyanoacrylate or anaerobic adhesive known in the art. Such a bond matrix 240 will provide suitable strength to resist tension, compression and torsional forces. To prevent excess bending forces from being applied to the bond matrix 240, the fracturable bond can be carried in within a male-female receiving structure. In this embodiment, the bond matrix 240 is carried within a receiving lumen of a coil portion that extends about a distal portion of the guide member as can be seen in FIG. 8C.

The detachment of the implant body 210 from the guide member by a pulse of coherent light from a laser. Such a light pulse can deliver energy very rapidly to a targeted media—i.e., the selectively absorbing nanocrystalline particles 244 described herein which comprise a chromophore. When the targeted particles are highly absorbing relative to a selected wavelength, the resulting photoabsorption causes thermoelastic expansion of the targeted nanoparticles and a rise in internal pressures within the particles. The term stress confinement refers to the process of causing this increase in pressure within a targeted media before the pressure can dissipate from the target at the speed of sound. When there exists a defined or free boundary between the targeted media and different surrounding media, such as a liquid or gas interface with the target, the target expands at its surface and then snaps back. The expansion phase is positive pressure or stress and the snap-back is negative stress. For example, a laser pulse can that can induce from 10° to 50° C. temperature rises in a targeted composition theoretically can cause transient pressures of from 100-1000 atmospheres within the targeted composition. This process of laser energy absorption in the targeted nanoparticles 144 can cause formation of a bipolar positive/negative stress wave that propagates into surrounding media. If the surrounding media were a liquid or gel, the bi-polar positive/negative stress wave would create a cavitation bubble within such media. In this case, the surrounding binder media 146 is a substantially solid material, and the stress wave causes a fracture or break in these materials called a spall plane. By this process, the thermoelastic expansion of the nanoparticles at the selected wavelength caused by a nanosecond laser pulse can yield a±10 atm (atmosphere) bipolar stress wave—and the −10 atm negative stress can easily cause a spall plane entirely across the thin bond matrix 240 this instantly detaching the previously connected terminal surfaces 236a and 238a of the guide member and implant body 210 (see FIG. 9).

In one embodiment, the nanocrystalline particles 244 have an average diameter less than about 500 nm, wherein the term average diameter means either a diameter of a substantially spherical nanoparticle or the principal (elongate) axis of a less spherical or non-spherical nanoparticle. More preferably, the nanoparticles 244 have an average diameter ranging from about 1 nm to 200 nm. The chromophore nanoparticles preferably have a uniformity of dimension, purity, and sphericity thus allowing a selected wavelength of light be absorbed uniformly by all particles. The preferred manner of fabricating the chromophore nanoparticles is again the laser pyrolysis method developed by NanoGram Corp.

For any selected wavelength, the chromophore is selected one the basis of its absorption coefficient so that it is strongly absorbing. The following sections describe exemplary chromophores that can comprise, or be carried by, the implantable nanometric particles of the invention and the spectral range for which they are best suited, commencing with chromophore that are strongly absorbing in preferred lower wavelength ranges from about 400 nm to 2000 nm. age 18

Biocompatible pure iron (Fe) can serve as a suitable chromophore. Iron can be fabricated into nanoparticles having uniform diameters of about 1 nm to 10 nm. For several reasons, the absorption coefficient (p6) peaks for iron-carrying nanoparticles are not certain, although estimates can be made since hemoglobin is a commonly targeted chromophore in photothermolysis techniques. Also, NanoGram Corp. has found that some nanoparticles (e.g., a titanium oxide (TiO₂)), when fabricated in particle sizes below a certain critical value, have an optical absorption band that shifts leading to different absorption peaks. This property can be useful to improve the performance of nanoparticles when functioning as a chromophore. It is not known at this time whether iron nanoparticles of the preferred dimensions will shift μₐ peaks, or why the μₐ shifts. The best estimates of the μₐ for Fe when taken from investigations of hemoglobin spectra are further complicated by the fact that values are typically tabulated by various “equivalents” that contain 1 gm atom of Fe that combines with 1 gm molecule of either O₂ or CO. In any event, such hemoglobin equivalents have one absorption peak at about 400 nm and another lower peak at about 520-550 nm. Using hemoglobin as a proxy for pure Fe chromophore nanoparticles np is still reasonable, and the method of the invention can generalize the use of wavelengths ranging from about 400 nm to 600 nm to absorb a laser pulse. NanoGram Corp. has also fabricated and characterized iron oxides nanocrystals and the use of any such iron oxides fall within the scope of the invention. It is believed that the peaks for such iron oxides will be similar to Fe but further testing is required. Carbon in the form of nanocrystalline particles having uniform diameters also can be used in about 1 nm to 100 nm dimensions. NanoGram Corp. has fabricated and characterized such carbon nanocrystals. The μₐ for carbon is believed to be without sharp peaks across the preferred spectrum with higher absorptions at shorter wavelengths, and is suitably absorbing up from 400 nm to 2000 nm. The method of the invention thus can generalize the use of wavelengths ranging from about 400 nm to 2000 nm to cause photomechanical energy effects in a selected nanoparticle composition that is strongly absorbing for that wavelength.

Now turning back to FIGS. 8A-8D, it can be seen how the functionality provided by the shape memory material is used to engage the rim wall rw portions and apply lateral or outward pressure on these wall portions. FIG. 8A shows the initial axial deployment of the implant body 210 from the distal end of catheter 205 in the direction of the arrow. In FIG. 8B, the implant body 210 is pushed further from lumen of catheter 205 and it can be seen that shape memory tensioning forces built into the implant body are released as the implant moves toward its second repose or expanded position. Of particular interest, by loading the medial portion 222 and the contramedial portions 252a and 252b with both bending and twisting deformations in moving the body 210 to the first linear shape for disposition in the catheter, the subsequent implant deployment can direct the implant body to apply lateral forces (substantially transverse to axis 255 of the catheter as shown by arrows) against the walls about circumference of the dome of the aneurysm an. FIG. 8C shows the deployment of implant body 210 before de-coupling as it engages the aneurysm sac somewhat above the narrow neck of the aneurysm. If the implant body 210 cannot be stabilized within the aneurysm, it can be withdrawn back into the catheter. FIG. 8D shows the implant body 210 in its final deployment after both ends 220A and 220B of the implant body 210 are simultaneously detached from guide members 230a and 320b.

After deployment of implant 210, the method of the invention is practiced as described previously wherein a charge source 120 carried by the implant causes charge-induced thrombosis. This implant 310 can carry the electroactive anode and cathode layers, and the exposed conductor, exactly as described in the Type “A” embodiment. The electroactive layers and conductor can be carried about the entire implant body, or any selected portion thereof. In one preferred embodiment, the electroactive layers and positive polarity conductor is carried at the end portions of the implant body that are deployed well within the aneurysm sac, and not at the medial portion of the implant that crosses the neck of the aneurysm.

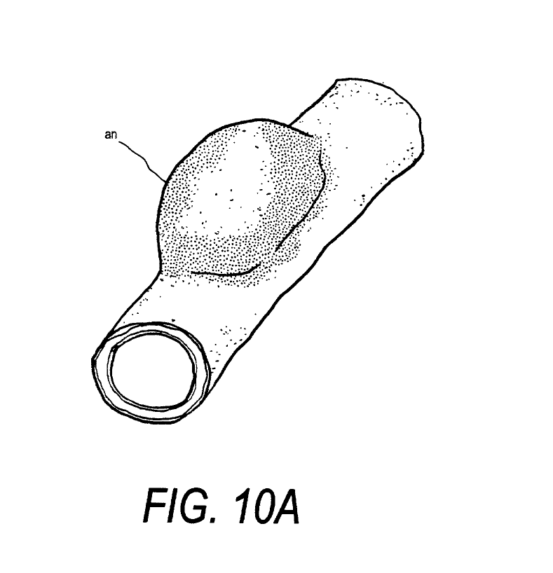

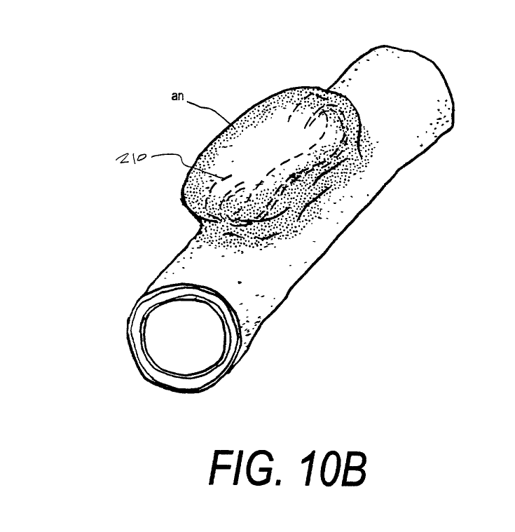

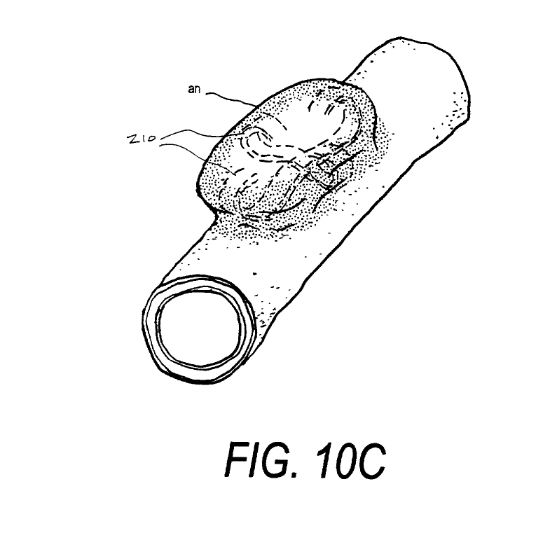

Turning now to FIGS. 10A-10C, other views of the vessel and aneurysm are shown to explain the method of the invention. FIG. 10A shows the narrow neck aneurysm from the exterior of the vessel, which is similar to the sectional views of the aneurysm in FIGS. 8A-8B. FIG. 10B shows the aneurysm an with implant 210 deployed which is similar to FIG. 8D. FIG. 10C shows two implant bodies 210 implanted in the aneurysm, and any number from one implant to several fall within the scope of the invention.

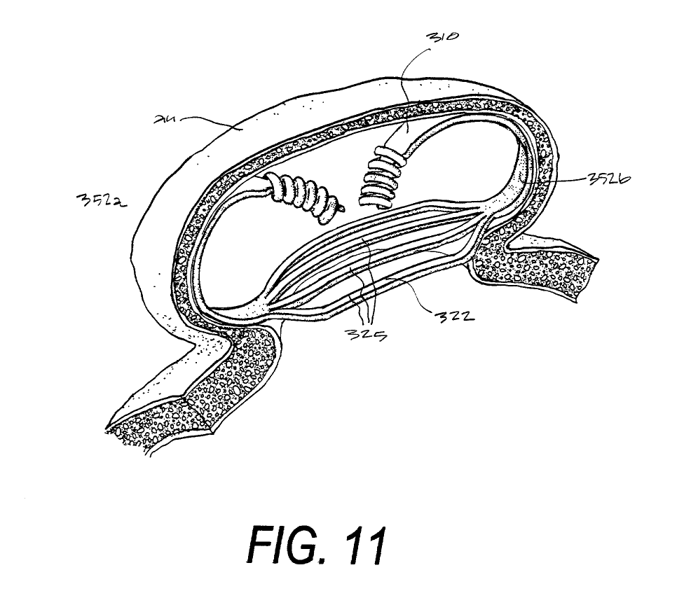

Now turning to FIG. 11, an alternative embodiment of implant body 310 is shown deployed in an aneurysm. This implant body is functionally equivalent to the previously described embodiment, with the only difference being an medial portion 322 that has multiple extending elements 325 (collectively) of a shape memory material that allows the medial portion to expand laterally in the second expanded position. This embodiment thus provides a grid of elements 325 that can substantially block the neck of the aneurysm after deployment. By deploying a grid across the neck, the lack of blood flow velocity through the grid and about the aneurysm sac can, at times, be sufficient to cause occlusion of the aneurysm. Another embodiment (not shown) has a similar medial portion 322 that comprises a collapsible-expandable mesh. The use of one or more implant bodies 210 or 310 as depicted in FIGS. 8D & 11 have the advantage that the aneurysm sac is not tightly packed with embolic material. Since the method of the invention utilizes a charge source 120 for charge-induced thrombosis, the aneurysm sac can substantially shrink in total volume over time as the patient's body absorbs the occluded aneurysm—which is not possible when the aneurysm sac is packed with embolic material such as a GDC coil. It should be appreciated that implant body portions between the medial portion 322 and the ends can have similar laterally expanding elements.

In another embodiment (not shown) the implant body can have a plurality of shape memory wires and be formed into a three-dimensional cage, with the electroactive layers of the invention carries about the distal portion of the cage, or the entire cage.

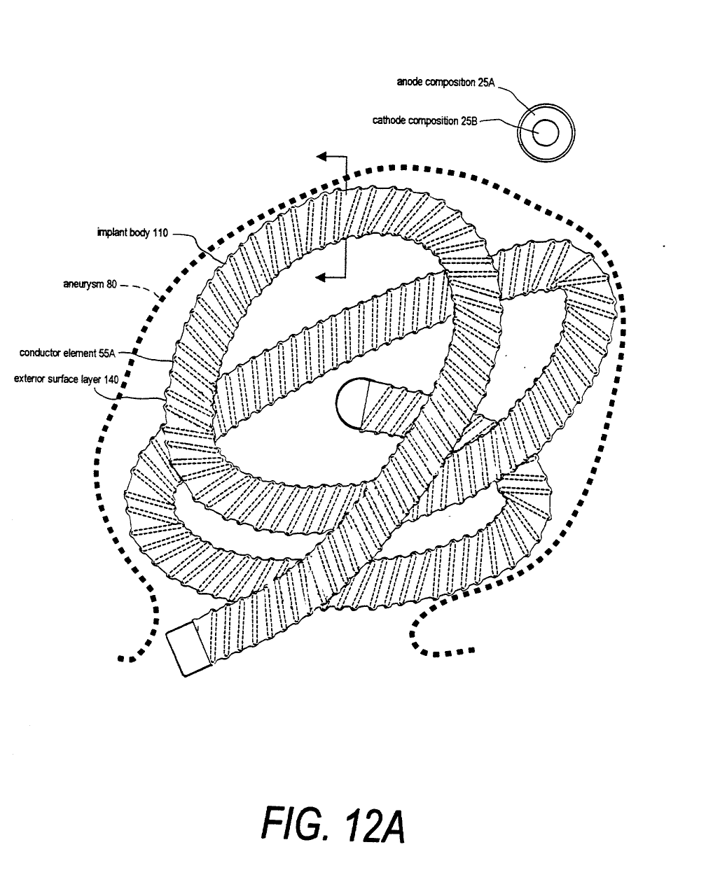

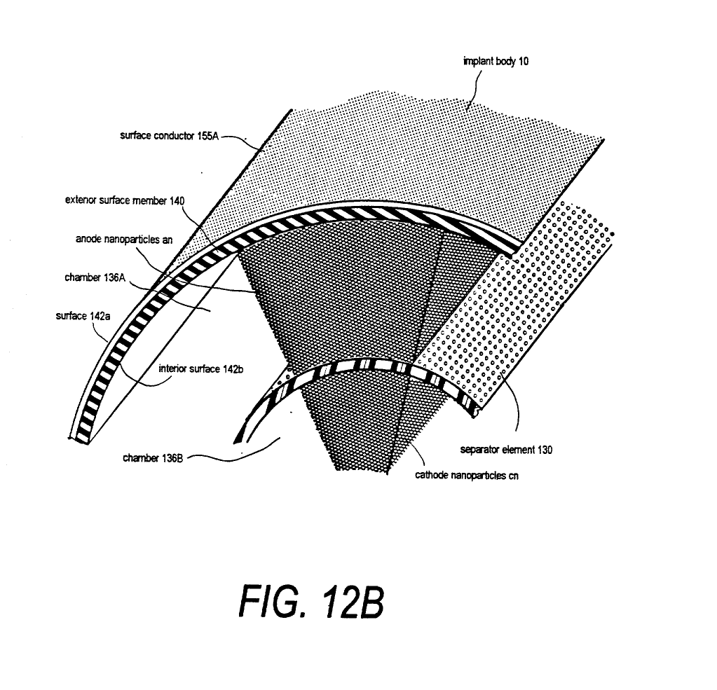

In another embodiment referring to FIGS. 12A-12B the anode and cathode layer can be carried in flexible polymer sleeve, with flexible layers of electroactive materials, thus allowing for greater volumes of such electroactive materials.

In another embodiment (not shown) the implant body can have a system including a capacitor for transient energy storage, a system for controlled leakage of a positive charge over time, and a system for coupling the charge or voltage source to the surface conductor only at the time of de-coupling. For example, the photoabsorption of a laser pulse can be adapted to couple (close a switch) the electrical source to the exposed surface conductor.

Those skilled in the art will appreciate that the exemplary embodiments and descriptions of the invention herein are merely illustrative of the invention as a whole. Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only being the true purview, spirit and scope of the invention.

What is claimed is:

1. A vaso-occlusive device for treating a vascular malformation, comprising:
an implantable body defining a volume and dimensions suited for implantation at a targeted site in the patient's vasculature; and
an electrical charge source carried by the said body capable of exposing a selected charge to endovascular media after implantation.

2. The device of claim 1 wherein the electrical charge source comprises a volume of electroactive particles carried by portions of said implantable body.

3. The device of claim 4 wherein said volume of electroactive particles is carried in at least one substantially thin layer around said implantable body.

4. The device of claim 4 wherein said volume of electroactive particles is carried in an interior layer of said implantable body.

5. The device of claim 4 further comprising an exposed conductive element coupled to said volume of electroactive particles.

6. The device of claim 1 wherein the implantable body comprises at least one elongate wire-like member for deformable placement is an aneurysm sac.

7. The device of claim 1 wherein the implantable body comprises a plurality of wire-like members forming a three-dimensional structure for placement is an aneurysmal sac.

8. The device of claim 1 wherein the implantable body is of shape memory material having a first linear or collapsed shape for disposition in the guide wire lumen of a catheter and a second expanded shape for disposition in an aneurysm and said implantable body has first and second end portions that are detachably coupled to first and second guide members.

9. A vaso-occlusive method, comprising the steps of:
implanting an implant body in a targeted site in a patient's vasculature, wherein the implant body carries a self-contained electrical charge source; and
exposing body media within the targeted site to a selected electrical charge thereby enhancing the formation of thrombus in the targeted site.

10. The vaso-occlusive method of claim 9 wherein the electrical charge is positive.

11. The vaso-occlusive method of claim 9 wherein the electrical charge is negative.

12. The vaso-occlusive method of claim 9 wherein the electrical charge is both positive and negative thereby causing current flow in the endovascular media.

13. A vaso-occlusive device for treating a vascular malformation, comprising:
an implant body defining a length and cross-section suitable for placement in a vascular malformation; and
a voltage source carried within the implantable member.

14. The vaso-occlusive device of claim 13 wherein the voltage source comprises an anode and a cathode of electroactive compositions and a separator element disposed therebetween.

15. The vaso-occlusive device of claim 14 wherein said electroactive compositions comprise nanoparticles.

16. The vaso-occlusive device of claim 15 wherein said nanoparticles have an average cross-section ranging from about 1 nm to 250 nm.

17. The vaso-occlusive device of claim 13 further comprising a first conductive material coupled to said anode composition and exposed to an exterior of the body.

18. The vaso-occlusive device of claim 13 further comprising a second conductive material coupled to said cathode composition and exposed to an exterior of the body.

19. The vaso-occlusive device of claim 13 wherein the voltage source provides from about 0.01 to 5 volts.

20. The vaso-occlusive device of claim 13 further comprising;
a detachable coupling wherein said vaso-occlusive device has a first end surface and a guide member has a second end surface, the first and second surfaces adhered together by a bond matrix;
wherein the guide member comprises an optic fiber; and
wherein the bond matrix carries a volume of chromophore particles that thermoelastically expand upon photoabsorption of a selected wavelength delivered through the optic fiber to propagate a bi-polar stress wave within the bond matrix.
